(19) United States
(12) Patent Application Publication
(10) Pub. No.: US 2015/0216699 A1
(43) Pub. Date: Aug. 6, 2015

(54) HEMORRHOIDAL DEVICE

(71) Applicant: Finn Kobke, (US)

(72) Inventor: Finn Kobke, Farevejle (DK)

(21) Appl. No.: 14/426,090

(22) PCT Filed: Sep. 11, 2013
(86) PCT No.: PCT/DK2013/050293

§ 371 Date: Mar. 4, 2015

(30) Foreign Application Priority Data
Sep. 14, 2012 (DK) PA 2012 70565

(51) Int. Cl. A61F 5/00

(52) U.S. Cl. CPC A61F 5/0093

(57) ABSTRACT

A device for the treatment of hemorrhoids. The device has a body with an external surface, and comprises an elastic and pliable material, allowing said body to be bent and inserted between the buttocks of a patient, so that the external surface can support any hemorrhoids in contact with the external surface, while the tension of said body allows the device to be held in place by the buttocks, while said body remains in a bent state. The device can be used for the treatment of hemorrhoids, fecal incontinence, flatulence or rectal prolapse.

The present invention relates to a device for the treatment of hemorrhoids. In particular, the present invention relates to an elastic, pliable device, which may be inserted in between the buttocks for the treatment of hemorrhoids, fecal incontinence, flatulence and/or rectal prolapse.

### TECHNICAL BACKGROUND

According to Wikipedia, hemorrhoids are vascular structures in the anal canal which help with stool control. They become pathological or piles when swollen or inflamed. In their physiological state, they act as a cushion composed of arteriovenous channels and connective tissue that aid the passage of stool.

Recommended treatment consists of increasing fiber intake, oral fluids to maintain hydration, NSAID analgesics, sitz baths, and rest. Surgery is reserved for those who fail to improve following these measures.

There are two types of hemorrhoids, external and internal, which are differentiated via their position with respect to the dentate line. Internal hemorrhoids are those that occur above the dentate line. Hemorrhoids can be further graded by the degree of prolapse.
Grade I: No prolapse. Prominent blood vessels may be present.
Grade II: Prolapse upon defecation but spontaneously reduce.
Grade III: Prolapse upon defecation and must be manually reduced.
Grade IV: Prolapsed and cannot be manually reduced.

The U.S. Pat. No. 4,583,542 describes a hemorrhoidal device insertable in to the rectum for treatment of hemorrhoids by the application of gentle pressure against the dilated veins. The device includes a body of soft pliable plastic having a tapered head portion with a circumferential shoulder which terminates at the shaft portion of the device, the end of the shaft portion terminating in an outwardly standing flange. Concentrically molded within the body of the device is a stiffener member extending the substantial length of the device for providing sufficient rigidity for insertion and tensile strength for removal.

The U.S. patent application U.S. 2004/0167479 A1 describes a hemorrhoid treatment pad, the hemorrhoid treatment pad having a substance on a body facing surface thereof, the substance comprising a skin care composition, wherein the skin care composition can have from about 0.001% to about 0.1% by weight of hexamidine; from about 0.001% to about 10% by weight of zinc oxide; from about 0.01% to about 10% by weight of niacinamide; and a carrier.

### SUMMARY OF THE INVENTION

There is a continued need for alternative treatments of hemorrhoids. In particular, there is a need for an inexpensive device which may win popular acceptance among patients. In spite of widespread prevalence of hemorrhoids in the population, the ailment is associated with embarrassment, leaving many patients untreated. Thus, there is a need for a device, which may be handled by a patient in a safe manner without the need of a professional practitioner. Accordingly, there is a need for a device, which may be sold over the counter (OTC).

According to an aspect, the present invention concerns a device comprising a body, said body having at least one external surface; said body comprising an elastic and pliable material, allowing said body to be bent and inserted between the buttocks of a patient; allowing said at least one external surface to support any hemorrhoids in contact with said at least one external surface, while the tension of said body allows said body to be held in place by the buttocks, while said body remains in a bent state. The device is preferably inserted such that contact with the anus and/or any prolapsed hemorrhoids is established.

Surprisingly, the present device has shown remarkably results in treating hemorrhoids, and on some occasions rendered surgery unnecessary.

In addition, the present invention does not need to penetrate in between the sphincter muscles, but may support hemorrhoids from the outside of the anal canal.

According to another aspect, the present invention concerns a method of treatment of hemorrhoids comprising insertion of a device according to the invention in between the buttocks.

According to an aspect, the present invention concerns a method of alleviating pain caused by hemorrhoids, preferably before an operation.

### DETAILED DISCLOSURE

Certain embodiments of the invention are described below, in the claims and in the figures to further illustrate the invention.

According to an embodiment, the present invention concerns a device comprising a body, said body having at least one external surface; said body comprising an elastic and pliable material, allowing said body to be bent and inserted between the buttocks of a patient; allowing said at least one external surface to support any hemorrhoids in contact with said at least one external surface, while the tension of said body allows said body to be held in place by the buttocks, while said body remains in a bent state. The device is preferably inserted such that contact with the anus and/or any prolapsed hemorrhoids is established. The device is preferably a hemorrhoidal device. Alternatively, the device may be a fecal incontinence device, a flatulence device and/or a rectal prolapsed device.

The patient may be an animal or human. The device is particularly suitable for a human patient.

Surprisingly, such a device has shown remarkably results in treating hemorrhoids, on some occasions replacing surgery. Further, the device may be helpful for subjects having problems controlling feces, fecal incontinence or flatulence.

The elasticity of the body provides tension upon bending the body.

For the present device no penetration through the anal canal into the rectum is necessary, as the elasticity of the body may keep the device in place. Known devices penetrate the anal canal and are held in place inter alia by sphincter muscles, which may create discomfort for the patient or user.

The elasticity of the body allows the at least one external surface to support any hemorrhoids sufficiently to at least partly counteract any prolapse and/or prominent blood vessels due to the tension induced by the bending of the body.

The present device does not need any particular skin care composition to support or treat hemorrhoids.

According to an embodiment, the present invention concerns a device, wherein said body has an upper side and a lower side; and wherein the pliability of said body allows said body to be shaped, the upper side convexly, the lower side concavely, by bending said body. The body is preferably bendable with the fingers.

When reference is made to an “upper side”, the upper side comprises at least one external surface for supporting hemorrhoids. In other words, a physical interface of the body of the device, allowing establishing contact with the anus and/or hemorrhoids, is situated on the upper side.

The terms “convexly” and “concavely” are used here to explain how the body is allowed to be shaped, and does not necessarily convey the meaning of perfectly convex or concave, but rather indicate the direction of the bend of the body. In line with this, according to an embodiment, the body may be a disc, which is bent in the middle, upon insertion, along a line crossing the disc, such that edges or peripheral parts of the lower side approach each other.

According to an embodiment, the present invention concerns a device, wherein said body is flat or substantially flat, and has an upper side and an opposing lower side, and has at least one edge between said upper side and said lower side; allowing said body to be shaped, the upper side convexly, the lower side concavely, by bending said body.

According to an embodiment, the present invention concerns a device, wherein said body has a central part and at least one peripheral part, and wherein said central part is thicker than said at least one peripheral part.

The at least one peripheral part, which is preferably thinner than the central part, allows close contact with the buttocks of the patient. This means the device may stay in place even if the patient moves around, due to the pressure from the buttocks.

Another advantage associated with a thicker central part is that the thicker part may provide increased pressure against the anus and any prolapsed hemorrhoids. The thicker part may additionally increase the outward pressure provided by the at least one peripheral part.

According to an embodiment, the present invention concerns a device, wherein said peripheral part constitutes at least 20% of said body when viewed from the upper side, the average thickness of said peripheral part being less than 50%, preferably less than 40%, more preferred less than 30%, preferably less than 20% of the average thickness of said central part.

According to an embodiment, the present invention concerns a device, wherein said peripheral part constitutes at least 30% of said body when viewed from the upper side, the average thickness of said peripheral part being less than 50%, preferably less than 40%, more preferred less than 30%, preferably less than 20% of the average thickness of said central part.

According to an embodiment, the present invention concerns a device, wherein said body comprises a central part and at least two peripheral parts adjacent to said central part; wherein said central part is thicker than said peripheral parts adjacent to said central part; wherein said central part is elongated; and wherein said central part is positioned between said at least two peripheral parts adjacent to said central part, allowing said two peripheral parts adjacent to said central part to approach each other upon insertion of said body in between the buttocks.

According to an embodiment, the present invention concerns a device, wherein said body has at least one edge or circumference, and wherein said body is tapered from the central part towards said edge or circumference.

According to an embodiment, the present invention concerns a device, wherein the body has an upper side, wherein the shape of the outline of the surface of said upper side is selected among circular, elliptic, quadratic, parallelogram shaped, quadrangle shaped, quadratic with rounded corners, parallelogram shaped with rounded corners, and quadrangle shaped with rounded corners; or substantially one of these shapes, before said body is bent.

According to an embodiment, the present invention concerns a device, wherein said body has an upper side, wherein the central part of said upper side is flat before said body is bent.

According to an embodiment, the present invention concerns a device, wherein said body has an upper side which has a recess in the central part, before said body is bent, facilitating bending of said body.

According to an embodiment, the present invention concerns a device, wherein said body has a lower side; wherein the central part of said lower side protrudes out of the body, before said body is bent.

According to an embodiment, the present invention concerns a device, wherein said body has a lower side, wherein the central part of said lower side is convex before said body is bent.

According to an embodiment, the present invention concerns a device, wherein said body has an upper side and a lower side, wherein the surface of at least one of said upper side and said lower side is smooth, preferably the surface of said upper side is smooth. A smooth surface may improve the adherence of the device to the end of the anal canal and/or the buttocks. Without being bound by theory, it is speculated that upon use of a device according to the invention, a vacuum may be established within the anal canal improving adherence of the device.

According to an embodiment, the present invention concerns a device, wherein said body has an upper side and a lower side, wherein the surface of at least one of said upper side and said lower side is rough; preferably the surface of said lower side is rough. A rough surface may facilitate handling of the device, e.g. with the fingers, making placement and subsequent removal easier. The word rough is used to express unsmooth.

According to an embodiment, the present invention concerns a device, wherein said body is made of a material selected among rubber, such as natural rubber, silicone, and resin. The body preferably has the elasticity and/or pliability of natural rubber, but other materials may be preferred. As an alternative, the body may comprise a pliable, non-elastic material combined with elastic stiffeners, such that the elastic stiffeners provide elasticity to the entire body. Further, the material used for the body is preferably selected in order to avoid adverse effects or reactions with a subject or patient using the device.

The body may comprise a material made of rubber, such as natural rubber or synthetic rubber. Candidates for a suitable rubber comprise, but are not limited to: Styrene Butadiene Rubber (SBR), Chloroprene Rubber (CR), (Acrylo) Nitrile Butadiene Rubber (NBR), Iso Butylene Isoprene (Butyl)

Rubber (IIR), Ethylene Propylene Rubber (EPDM or EPR), and Silicone Rubber. A lengthier list of potential candidates for a material suitable for the body comprise the group consisting of: Polyacrylate Rubber, Ethylene-acrylate Rubber, Polyester Urethane, Bromo Isobutylene Isoprene, Polybutadiene, Chloro Isobutylene Isoprene, Polychloroprene, Chlorosulphonated Polyethylene, Epichlorohydrin, Ethylene Propylene, Ethylene Propylene Diene Monomer, Polyether Urethane,

Perfluorocarbon Rubber, Fluoronated Hydrocarbon, Fluoro Silicone, Fluorocarbon Rubber, Hydrogenated Nitrile Butadiene, Polyisoprene, Isobutylene Isoprene Butyl, Acrylonitrile Butadiene, Polyurethane, Styrene Butadiene, Styrene Ethylene Butylene Styrene Copolymer, Polysiloxane, Vinyl Methyl Silicone, Acrylonitrile Butadiene Carboxy Monomer, Styrene Butadiene Carboxy Monomer, Thermoplastic Polyether-ester, Styrene Butadiene Block Copolymer, and Styrene Butadiene Carboxy Block Copolymer. Other materials may be suitable in this context.

According to an embodiment, the present invention concerns a device, wherein said body has the elasticity of natural rubber. According to an embodiment, the present invention concerns a device, wherein said body has the pliability of natural rubber. These embodiments mean that there is no need for an adhesive to keep the device in place.

The elasticity is determined by two types of material parameter. The first type is called a modulus, which measures the amount of force per unit area (stress) needed to achieve a given amount of deformation. The units are Pascals (Pa) or pounds per square inch (psi). The second type of parameter measures the elastic limit. The limit can be a stress beyond which the material is no longer elastic or a deformation beyond which elasticity is lost.

Rubber has a low modulus of elasticity and is capable of sustaining a deformation of as much as 1000 percent. After such deformation, it quickly and forcibly retracts to its original dimensions. Further details of the mechanical properties of rubber may be found in “Harris' Shock and Vibration Handbook”, fifth edition, Cyril M. Harris and Allan G. Piersol, chapter 33 by Ronald j. Schaefer. This reference further explains how to measure the tensile maximum and the elongation maximum of a material. The tensile maximum of natural rubber is indicated to be 4500 psi, while the elongation maximum is indicated to be 650%.

Rubber typically has a Young's Modulus (Modulus of Elasticity) in the range of 0.01-0.1 GPa. Young's modulus can be used to predict the elongation or compression of an object as long as the stress is less than the yield strength of the material.

According to an embodiment, the present invention concerns a device, wherein said body has a Modulus of Elasticity of less than 2 GPa, preferably less than or equal to 1 GPa, more preferred less than 0.75 GPa, preferably less than 0.5 GPa, more preferred less than 0.4 GPa, preferably less than 0.3 GPa, more preferred less than 0.2 GPa, preferably less than 0.1 GPa, more preferred less than 0.09 GPa, preferably less than 0.08 GPa, more preferred less than 0.07 GPa, preferably less than 0.06 GPa, more preferred less than 0.05 GPa, preferably less than 0.04 GPa, more preferred less than or equal to 0.03 GPa.

According to an embodiment, the present invention concerns a device, wherein said body has a Modulus of Elasticity of 0.001-2 GPa, preferably 0.002-1 GPa, more preferred 0.003-0.5 GPa, preferably 0.004-0.3 GPa, more preferred 0.005-0.2 GPa, preferably 0.006-0.1 GPa, more preferred 0.007-0.075 GPa, preferably 0.008-0.05 GPa, more preferred 0.009-0.04 GPa, preferably 0.01-0.03 GPa.

According to an embodiment, the present invention concerns a device, wherein said body has a tensile maximum of 100-50000 psi, preferably 500-10000 psi, more preferred 1000-7500 psi, preferably 1500-5000 psi, more preferred about 4500 psi.

According to an embodiment, the present invention concerns a device, wherein said body has an elongation maximum of at least 100%, preferably more than 100%, more preferred more than 200%, preferably more than 300%, more preferred more than 400%, preferably more than 500%, more preferred more than 600%, preferably more than 700%, more preferred more than 800%, preferably more than 900%, more preferred more than 1000%.

According to an embodiment, the present invention concerns a device, wherein said body has an elongation maximum of 100-2000%, preferably 200-1500%, more preferred 300-1200%, preferably 400-1000%, more preferred 500-900%, preferably 600-800%.

According to an embodiment, the present invention concerns a device, wherein said body is non-absorbent. A non-absorbent body may be preferable for hygienic reasons. Further, a non-absorbent body may make the device re-usable. The device of the present invention is preferably not absorbent. Using non-absorbent materials allows for multiple uses of the present device after cleaning. According to an embodiment, the present invention concerns a device, which is re-usable.

According to an embodiment, the present advice may be used more than one time, e.g. after proper cleaning.

According to an embodiment, the present invention concerns a device, which is specifically adapted for the treatment of hemorrhoids.

According to an embodiment, the present invention concerns a device, wherein said body is impervious to liquids. This allows the device to be re-usable.

According to an embodiment, the present invention concerns a device, wherein said body is water repulsive.

According to an embodiment, the present invention concerns a device, wherein said body is hydrophobic.

According to an embodiment, the present invention concerns a device, wherein said device is made of a homogenous material. The absence of a core of a different material lowers production costs.

According to an embodiment, the present invention concerns a device, wherein said device is massive.

According to an embodiment, the present invention concerns a device, wherein said body incorporates an active ingredient, e.g. an antibacterial agent, optionally as part of a gel.

According to an embodiment, the present invention concerns a device which is free of seams. Seams described in the prior art may be provided by means such as, but not limited to, gluing, crimping and heat-sealing. The absence of seams lowers production costs and removes any adverse effects associated with seams, such a irritation of the buttocks.

According to an embodiment, the present invention concerns a device, wherein said device has a density of about 1 g/cm³ or larger. The density is measured as the total mass of the device divided by the outer volume of the device. In order to measure the outer volume of e.g. an absorbent pad, the pad should be covered with a water impervious material such as a foil. A density of about 1 g/cm³ or larger means the device is easy to discard, as it provides improved sink properties without the need for the device to absorb water.

According to an embodiment, the present invention concerns a device, wherein the hemorrhoids to be treated are external or internal.

According to an embodiment, the present invention concerns a device according to any of the preceding claims, wherein the hemorrhoids have a grade selected among the group consisting of grade I, grade II, grade III, and grade IV; preferably the hemorrhoids have a grade selected among grade III and grade IV.

According to an embodiment, the present invention concerns a method of treatment of hemorrhoids comprising insertion of a device according to the invention in between the buttocks.

According to an embodiment, the present invention concerns a method of alleviating pain caused by hemorrhoids, preferably before an operation, by the use of a device according to the invention.

According to an embodiment, the present invention concerns a method of treatment of hemorrhoids, fecal incontinence, flatulence and/or rectal prolapse comprising use of a device according to the invention. This method preferably comprises insertion of the device in bent state in between the buttocks.

### FIGURES

FIG. 1 shows a photograph of a device (101) according to a preferred embodiment of the invention taken from the lower side of the body. The device was photographed with a background having horizontal and vertical lines. The spacing between these lines is 0.5 cm. Thus, the distance from the left side to the right side of the device on this photograph is about 6.5 cm. The distance from the top to the bottom of the device on this photograph is about 5.5 cm. This embodiment was made from natural rubber. The thicker, central part (103) of the body allows little light to pass through, while the thinner outer parts shown to the left (105) and right (107) of the central part are more transparent to visible light. This preferred embodiment has a shape reminding of a stingray (fish), thin in the two peripheral parts, left and right, and thicker in the central part of the body. Upon use, the two peripheral parts, or flaps, supports the device against the inner part of the buttocks, and supports the device up against the end of the anal canal.

FIG. 2 shows a similar photograph of a device according to an embodiment of the invention from the lower side of the body. This photograph is taken with a slanted angle with respect to the surface of the lower side of the body.

FIG. 3 shows a schematic drawing of an embodiment of a device (301) according to the invention from below. Dimensions are provided in millimeters. This device comprises a central part (303) and thinner, outer parts, one above the central part on the drawing, and one part (305) below the central part on the drawing.

FIG. 4 shows a schematic drawing of an embodiment of a device (401) according to the invention.

FIG. 4 is a cross section of FIG. 3, along line A-A of FIG. 3. Dimensions are provided in millimeters. Along the A-A axis is a recess (409) in the body, facilitating bending of the device.

FIG. 5 shows a schematic drawing of an embodiment of a device (501) according to the invention. FIG. 5 is a cross section of FIG. 3, along line C-C of FIG. 3. Dimensions are provided in millimeters. On FIG. 5, a central part (503) is shown, with one thinner outer part above, and one thinner outer part (505) shown below. The body has a recess (509) facilitating bending of the device.

FIG. 6 shows a simplified anatomical cross section of the anal canal (32) connected to the rectum (34). Located in the anal canal (32) are dilated veins (36), commonly referred to as hemorrhoids or piles. Located at the top of the anal canal (32) is the internal sphincter muscle (38) and the external sphincter muscle (40), the latter of which is voluntary while the prior is involuntary.

FIG. 7 shows a similar simplified anatomical cross section with an embodiment of a device (701) according to the invention in place. The device comprises a central part (703) and outer parts to the left (705) and right. The tension of the bent device keeps the device in place, supporting the hemorrhoids.

FIG. 8 shows a schematic drawing depicting the outline of an embodiment of a device according to the invention. The device is shown as seen from above in a non-bended, relaxed state.

FIG. 9 shows a schematic drawing depicting a cross-section of an embodiment of a device according to the invention. The device is depicted in a non-bended, relaxed state. The flat side with the recess is on the upper side on the drawing.

FIG. 10 shows a schematic drawing depicting a cross-section of an embodiment of a device according to the invention. The device is depicted in a bended state, adopted e.g. by bending it with two fingers before insertion between the buttocks. Upon inserting the device, hemorrhoids may be pushed up. After insertion, the outer, peripheral parts will exert pressure against the buttocks. The device will exert a pressure up against any hemorrhoids if contact has been established. The thicker, central part acts as a spring, aiding in pushing the outer, peripheral parts against the buttocks, allowing the device to be pushed upwards towards the anus when the patients moves.

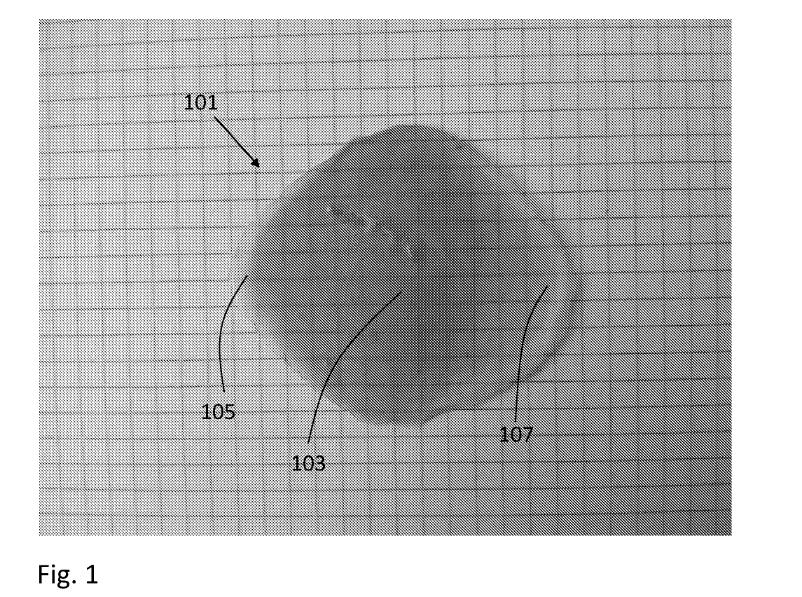

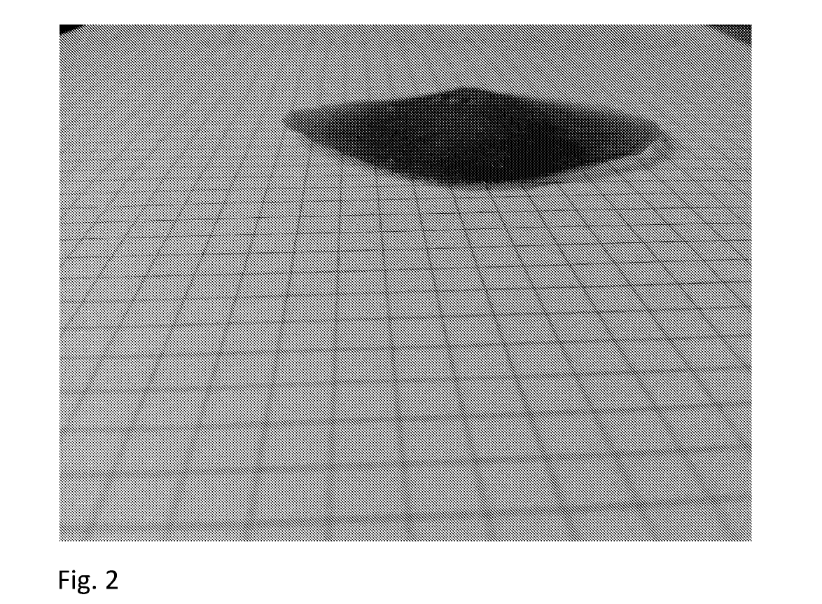

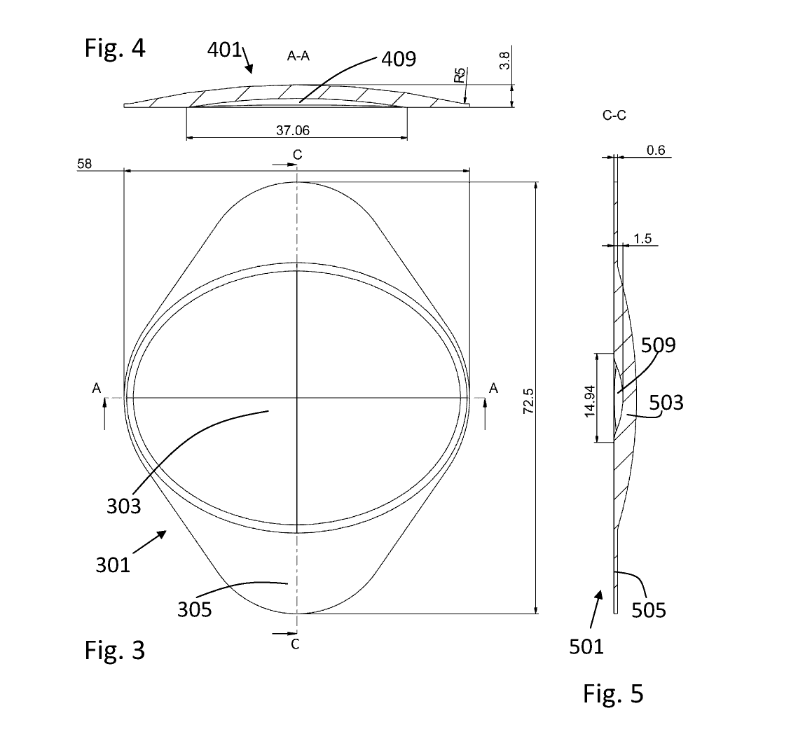

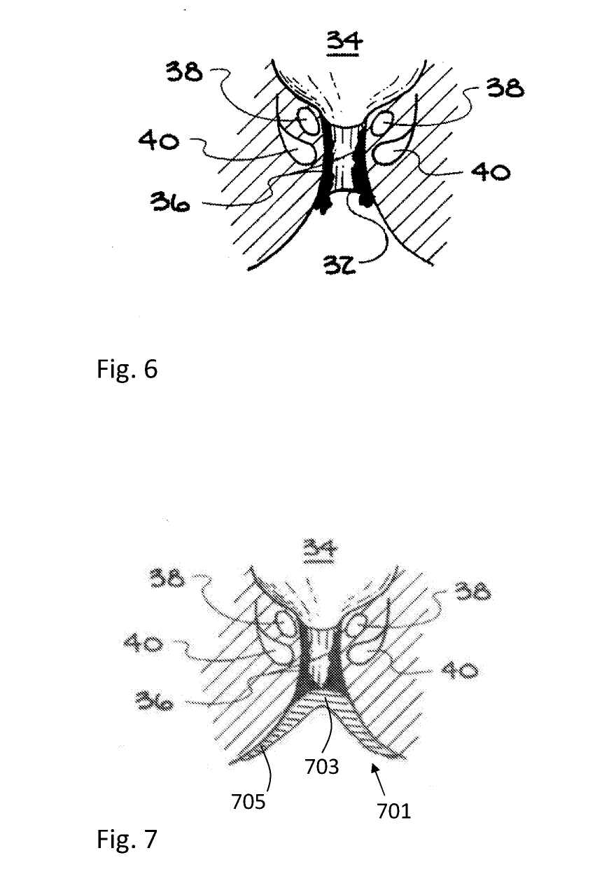

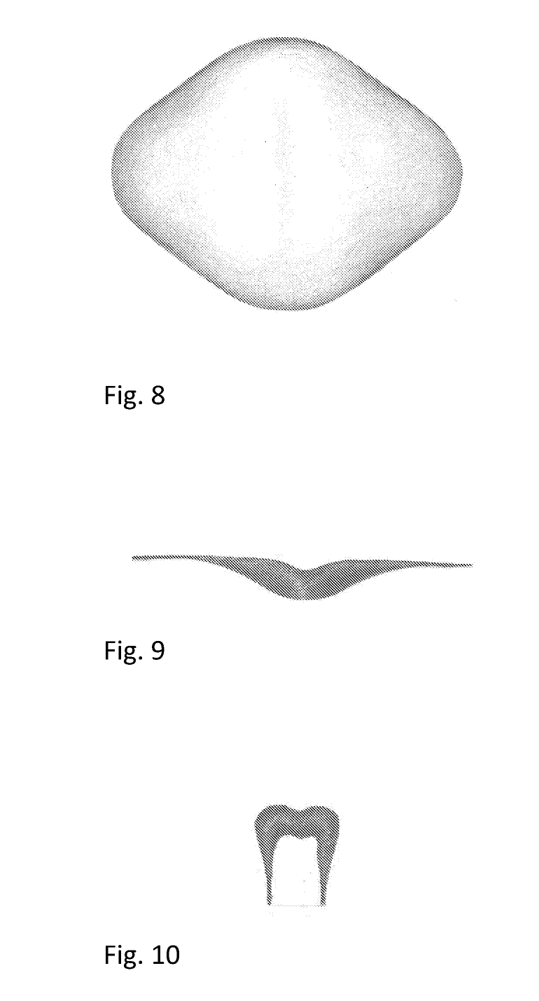

All cited references are incorporated by reference.

The accompanying Figures and Examples are provided to explain rather than limit the present invention. It will be clear to the person skilled in the art that aspects, embodiments and claims of the present invention may be combined.

### EXAMPLES

Subjects suffering from hemorrhoids of grade III and IV tested embodiments of the present invention. Good patient compliance was reported, and the ailment disappeared after short use.

1. A device having a body comprising an elastic and pliable material, allowing said body to be bent and inserted between buttocks of a patient, said body having an external surface;
with said external surface configured to support any hemorrhoids in contact with said external surface, while the tension of said body allows said body to be held in place by the buttocks, while said body remains in a bent state.

2. A device according to claim 1, which is a hemorrhoidal device.

3. A device according to claim 1, wherein said body has an upper side and a lower side; and wherein the pliability of said body allows said body to be shaped, the upper side convexly, the lower side concavely, by bending said body.

4. A device according to claim 1, wherein said body is flat or substantially flat, and has an upper side and an opposing lower side, and has at least one edge between said upper side and said lower side; allowing said body to be shaped, the upper side convexly, the lower side concavely, by bending said body.

5. A device according to claim 1, wherein said body has a central part and at least one peripheral part, and wherein said central part is thicker than said at least one peripheral part.

6-7. (canceled)

8. A device according to claim 1, wherein said body comprises an elongated central part and at least two peripheral parts adjacent to said central part;
wherein said central part is thicker than said peripheral parts adjacent to said central part; and
wherein said central part is positioned between said at least two peripheral parts, allowing said two peripheral parts to approach each other upon insertion of said body in between the buttocks.

9. A device according to claim 1, wherein said body has at least one edge or circumference, and wherein said body is tapered towards said edge or circumference.

10-11. (canceled)

12. A device according to claim 1, wherein said body has an upper side with a recess in the central part, before said body is bent, facilitating bending of said body.

13. A device according to claim 1, wherein said body has a lower side; and
wherein a central part of said lower side protrudes out of the body, before said body is bent.

14-15. (canceled)

16. A device according to claim 1, wherein said body comprises rubber, silicone, or resin.

17. A device according to claim 1, wherein said body has a Modulus of Elasticity of less than 2 GPa.

18. A device according to claim 17, wherein said body has a Modulus of Elasticity of 0.002-1 GPa.

19. A device according to claim 1, wherein said body has a tensile maximum of 100-50000 psi.

20. A device according to claim 1, wherein said body has an elongation maximum of at least 100%.

21. (canceled)

22. A device according to claim 1, wherein said body has an elasticity of natural rubber.

23. A device according to claim 1, wherein said body is non-absorbent.

24. A device according to claim 1, which is re-usable.

25. (canceled)

26. A device according to claim 1, wherein said body is impervious to liquids.

27. A device according to claim 1, wherein said body is water repulsive.

28. A device according to claim 1, wherein said body is hydrophobic.

29-37. (canceled)
